(19) United States
(12) Patent Application Publication
(10) Pub. No.: US 2016/0151568 A1
(43) Pub. Date: Jun. 2, 2016

(54) SYSTEMS AND METHODS FOR DELIVERING A THERAPEUTIC AGENT USING MECHANICAL ADVANTAGE

(71) Applicant: Massachusetts Institute of Technology, Cambridge, MA (US)

(72) Inventors: J. Richard Gyory, Sudbury, MA (US); Alessandro Pizzochero, Chelmsford, MA (US)

(73) Assignee: Massachusetts Institute of Technology, Cambridge, MA (US)

(21) Appl. No.: 14/866,626

(22) Filed: Sep. 25, 2015

Related U.S. Application Data
(63) Continuation of application No. 14/164,942, filed on Jan. 27, 2014, now abandoned.
Application No. 14/164,942 is a continuation of application No. 13/102,657, filed on May 6, 2011, now abandoned.
(60) Provisional application No. 61/332,067, filed on May 6, 2010.

(51) Int. Cl. A61M 5/168; A61M 5/145

(52) U.S. Cl. CPC A61M 5/16877; A61M 5/145

(57) ABSTRACT

Devices and methods for delivering a therapeutic agent to a patient are disclosed herein. In one embodiment, a delivery system includes a reservoir containing a fluid and a fluid communicator in fluid communication with the reservoir. An actuator is coupled to the reservoir and configured to displace and exert a force on the reservoir for a time period upon actuation such that fluid within the reservoir is communicated through the fluid communicator. An amplification mechanism is coupled to the actuator. The amplification mechanism is configured to increase at least one of the force, the displacement, or the time period the force is exerted by the actuator on the reservoir. In some embodiments, a transfer structure is disposed between the amplification mechanism and the reservoir. The transfer structure is configured to contact the reservoir upon actuation of the actuator. In some embodiments, the actuator can be an electrochemical actuator.

### CROSS-REFERENCE TO RELATED APPLICATIONS

This application claims priority to and the benefit of U.S. Provisional Application Ser. No. 61/332,067, filed May 6, 2010, entitled “Systems And Methods For Delivering a Therapeutic Agent Using Mechanical Advantage,” the disclosure of which is hereby incorporated by reference herein in its entirety.

### BACKGROUND

The invention relates generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid some of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Further, modulating or controlling the delivery of drugs using such systems may not be possible without using complex systems.

Some infusion pump systems may be large and may require tubing between the pump and the infusion set, which can impact the quality of life of the patient. Further, infusion pumps may be expensive and may not be disposable. From the above, it would be desirable to provide new and improved drug delivery systems and methods that overcome some or all of these and other drawbacks.

### SUMMARY OF THE INVENTION

Devices and methods for delivering a therapeutic agent to a patient are disclosed herein. In one embodiment, a delivery system includes a reservoir containing a fluid and a fluid communicator in fluid communication with the reservoir. An actuator is coupled to the reservoir and configured to exert a force on the reservoir for a time period upon actuation such that fluid within the reservoir is communicated through the fluid communicator. An amplification mechanism is coupled to the actuator. The amplification mechanism is configured to increase at least one of the force, displacement, or the time period the force is exerted by the actuator on the reservoir. In some embodiments, a transfer structure is disposed between the amplification mechanism and the reservoir. The transfer structure is configured to contact the reservoir upon actuation of the actuator. In some embodiments, the actuator can be an electrochemical actuator.

### BRIEF DESCRIPTION OF THE DRAWINGS

FIG. 1 is a schematic illustration of a delivery system according to an embodiment.

FIG. 2A is a side view of a schematic illustration of an electrochemical actuator shown in a charged state; and FIG. 2B is a schematic illustration of a side view of the electrochemical actuator of FIG. 2A shown in a discharged state.

FIG. 3A is a schematic illustration of a portion of a delivery system according to an embodiment illustrating an electrochemical actuator in a charged state; and FIG. 3B is a schematic illustration of the portion of the delivery system of FIG. 3A illustrating the electrochemical actuator as it discharges.

FIG. 4A is a perspective view of a delivery system according to an embodiment and FIG. 4B is an exploded view of the delivery system of FIG. 4A.

FIG. 5A is a side view of a schematic illustration of a delivery system according to an embodiment, shown in a charged state and FIG. 5B is a side view of the delivery device of FIG. 5A shown in a discharged state.

FIG. 6 is a top view of a portion of the delivery system of FIGS. 5A and 5B.

FIG. 7 is a perspective view of an amplification mechanism according to another embodiment shown in an extended configuration.

FIG. 8 is a side view of the amplification mechanism of FIG. 7.

FIG. 9 is a top view of the amplification mechanism of FIG. 7.

FIG. 10 is a perspective view of the amplification of FIG. 7 shown in a collapsed configuration.

FIG. 11 is a side view of the amplification mechanism of FIG. 7 and a transfer structure according to an embodiment.

FIG. 12 is a perspective view of the amplification mechanism and transfer structure of FIG. 7 and a fluid source according to an embodiment.

FIG. 13 is a perspective view of an amplification mechanism according to another embodiment shown in an extended configuration.

FIG. 14 is a top view of the amplification mechanism of FIG. 13.

FIG. 15 is a side view of the amplification mechanism of FIG. 13 and a transfer structure according to an embodiment.

FIG. 16 is a perspective view of the amplification mechanism and transfer structure of FIG. 15 and a fluid source according to an embodiment.

FIG. 17 is a side view of the amplification of FIG. 13 shown in a collapsed configuration.

FIG. 18 is a perspective view of an amplification mechanism according to another embodiment.

FIG. 19 is a side view of an actuator according to an embodiment, shown in a discharged state.

FIG. 20 is a side view of the actuator of FIG. 19 and an amplification mechanism according to an embodiment, shown in a discharged state.

FIG. 21 is a perspective view of an amplification mechanism and actuator according to another embodiment, shown in an extended configuration.

FIG. 22 is a side view of the amplification mechanism and actuator of FIG. 21, shown in a collapsed configuration.

FIG. 23 is a side view of the amplification mechanism and actuator of FIG. 21, shown in an extended configuration.

### DETAILED DESCRIPTION

Devices, systems and methods are described herein that are configured for use in the delivery of therapeutic agents to a patient's body. Such therapeutic agents can be, for example, one or more drugs and can be in fluid form of various viscosities. In some embodiments, the devices and methods can include a pump device that includes an actuator, such as, for example, an electrochemical actuator, which can have characteristics of both a battery and a pump. Specifically, an electrochemical actuator can include an electrochemical cell that produces a pumping force as the cell discharges. Thus, the pump device can have relatively fewer parts than a conventional drug pump, such that the pump device is relatively more compact, disposable, and reliable than conventional drug pumps. Such drug delivery devices are desirable, for example, for use in delivery devices that are designed to be attached to a patient's body (e.g., a wearable device). These attributes of the pump device may reduce the cost and the discomfort associated with infusion drug therapy.

In some embodiments, such a pump device can be operated with, for example, a controller and/or other circuitry, operative to regulate drug or fluid flow from the pump device. Such a controller may permit implementing one or more release profiles using the pump device, including release profiles that require uniform flow, non-uniform flow, continuous flow, discontinuous flow, programmed flow, scheduled flow, user-initiated flow, or feedback responsive flow, among others. Thus, the pump device may effectively deliver a wider variety of drug therapies than other pump devices.

In some embodiments of a drug delivery system, an amplification mechanism is used in conjunction with an actuator to enhance the pumping force and/or displacement of the actuator. The use of an amplification mechanism can provide a mechanical advantage to the system operation. Such enhanced pumping features can further increase the variety of different types of drug therapies that can be delivered using a wearable drug delivery system.

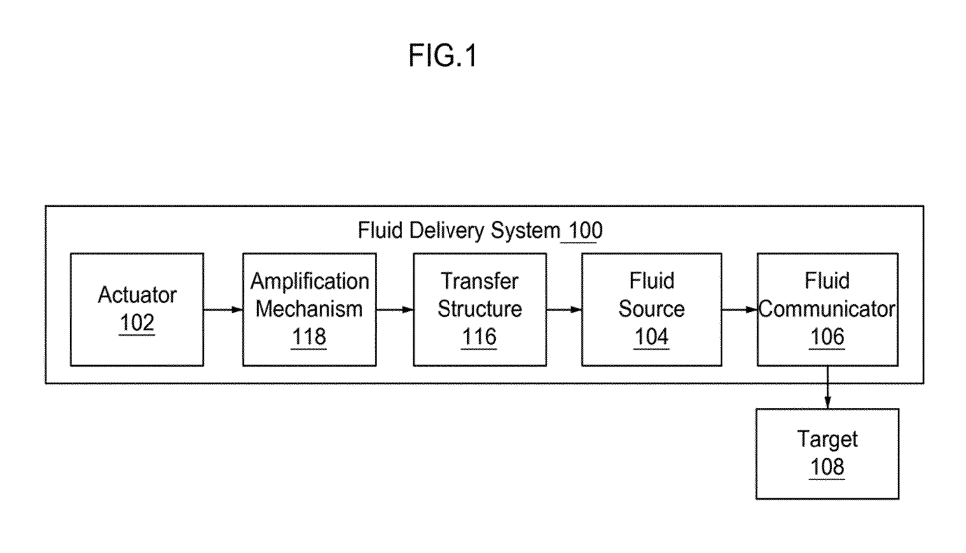

FIG. 1 is a schematic block diagram illustrating an embodiment of a fluid delivery system 100 (also referred to herein as “delivery device” or “drug delivery device”). The fluid delivery system 100 includes an actuator 102, an amplification mechanism 118, a transfer structure 116, a fluid source 104 and a fluid communicator 106. The fluid source 104 can contain a fluid (i.e., a therapeutic agent) to be delivered into a target 108 via the fluid communicator 106. The target 108 can be, for example, a human or other mammalian body in need of a drug therapy or prophylaxis.

The actuator 102 can be, for example, an electrochemical actuator 102 that can actuate or otherwise create a pumping force to deliver the fluid from the fluid source 104 into the fluid communicator 106 as described in more detail below. In some embodiments, the actuator 102 can be a device that experiences a change in volume or position in response to an electrochemical reaction that occurs therein. For example, the actuator 102 can be an electrochemical actuator that includes a charged electrochemical cell, and at least a portion of the electrochemical cell can actuate as the electrochemical cell discharges. Thus, the actuator 102 can be considered a self-powered actuator or a combination battery and actuator.

The amplification mechanism 118 can be used to enhance the pumping force and/or displacement of the actuator 102 (also referred to herein as the “stroke” of the actuator). For example, the overall displacement of the delivery system 100 can be increased by the use of the amplification mechanism 118 in conjunction with the electrochemical actuator 102. In some embodiments, the amplification mechanism 118 can be used to amplify the displacement along the major axis of displacement of the delivery device 100. For example, a typical electrochemical actuator can be substantially flat or planar in its undeformed or inactivated shape and then deforms in a direction substantially perpendicular to its flat configuration. When this type of actuator is coupled with a mechanical amplification mechanism, the overall motion or displacement can be increased.

In some embodiments, the amplification mechanism 118 can be used in conjunction with an actuator 102 that applies a relatively high force over a relatively short displacement. An actuator 102 having a short displacement and high force can be configured to perform a relatively large amount of work per volume of the actuator 102. Thus, the amplification mechanism 118 can be used to increase or decrease the displacement and/or increase or decrease the force to deliver a relatively large volume of fluid from the fluid source 104 per volume of the overall delivery system 100. For example, when used in conjunction with such an actuator 102, the amplification mechanism 118 can amplify the relatively short displacement of the actuator 102, but reduce the amount of force provided by the actuator 102. In some embodiments, the actuator 102 can be configured to provide a relatively large displacement, but a relatively small amount of force. Also, the amplification mechanism can be used to increase the force, but reduce the displacement. The amplification mechanism 118 can be configured to deliver different levels of force over different displacements depending on desired design parameters. Furthermore, the amplification mechanism 118 can be used to increase or decrease the overall time required to deliver a predetermined volume of fluid.

In some embodiments, the use of an amplification mechanism 118 can also change the duration of pumping force exerted by the actuator 102. For example, to pump a particular viscosity fluid out of a fluid reservoir, the amplification mechanism 118 may be configured to increase the displacement or stroke of the delivery system 100 and reduce the force exerted by the actuator 102, such that (1) the volume of fluid to be pumped can be increased without increasing the duration of the pumping, or (2) the volume of fluid is not changed, but the duration of pumping is reduced. In some embodiments, the amplification mechanism 118 may be configured to increase the force exerted by the electrochemical actuator 102 and decrease the displacement or stroke of the actuator 102. In such an embodiment, the increased force exerted can allow for a greater viscosity fluid to be pumped.

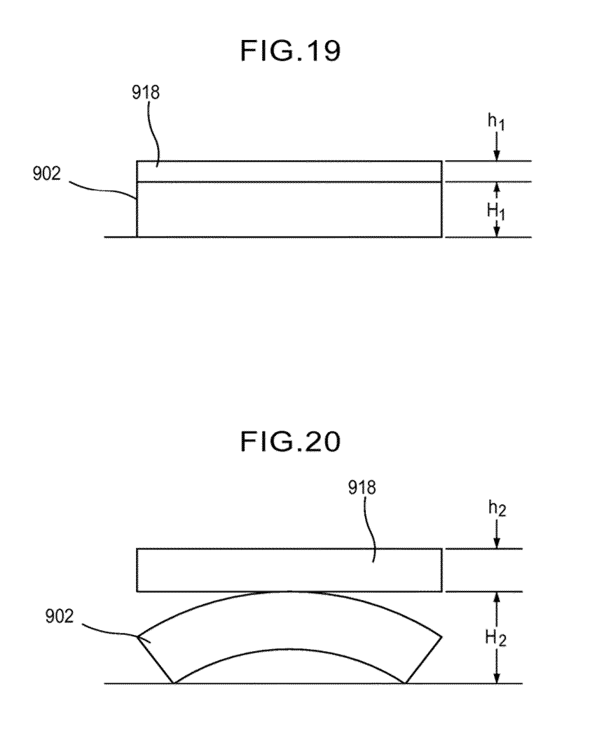

With increased displacement, and/or force, the delivery system 100 can be used to deliver a fluid volume that otherwise may not be possible without amplification. For example, with mechanical amplification, a drug delivery device can, in some embodiments, achieve a longer stroke than with no amplification (see, e.g., FIGS. 19 and 20). A longer stroke can be leveraged to deliver larger drug doses, thus enabling new therapies previously not possible with known wearable drug delivery devices. Specific embodiments of an amplification mechanism 118 are described in more detail below.

The fluid source 104 can be a reservoir, pouch, chamber, barrel, bladder, or other known device that can contain a drug in fluid form therein. The fluid communicator 106 can be in, or can be moved into, fluid communication with the fluid source 104. The fluid communicator 106 can be, for example, a needle, catheter, cannula, infusion set, or other known drug delivery conduit that can be inserted into or otherwise associated with the target body for drug delivery.

In some embodiments, the fluid source 104 can be any component capable of retaining a fluid or drug in fluid form. In some embodiments, the fluid source 104 may be disposable (e.g., not intended to be refillable or reusable). In other embodiments, the fluid source 104 can be refilled, which may permit reusing at least a portion of the device and/or varying the drug or fluid delivered by the device. In some embodiments, the fluid source 104 can be sized to correlate with the electrochemical potential of the electrochemical actuator 102. For example, the size and/or volume of the fluid source 104 can be selected so that the fluid source 104 becomes about substantially empty at about the same time that the electrochemical actuator 102 becomes about substantially discharged. By optimizing the size of the fluid source 104 and the amount of drug contained therein to correspond to the driving potential of the electrochemical actuator 102, the size and/or cost of the device may be reduced. In other embodiments, the electrochemical actuator 102 may be oversized with reference to the fluid source 104. In some embodiments, the delivery system 100 can include more than one fluid source 104. Such a configuration may permit using a single device to deliver two or more drugs or fluids. The two or more drugs or fluids can be delivered discretely, simultaneously, alternating, according to a program or schedule, or in any other suitable manner. In such embodiments, the fluid sources 104 may be associated with the same or different electrochemical actuators 102, the same or different fluid communicators 106, the same or different operational electronics, or the same or different portions of other components of the delivery system.

The transfer structure 116 can be disposed between the amplification mechanism 118 and the fluid source 104 or between the electrochemical actuator 102 and the fluid source 104. The transfer structure 116 includes a surface configured to contact the fluid source 104 upon actuation of the actuator 102 such that a force exerted by the electrochemical actuator 102 and/or the amplification mechanism 118 is transferred from the transfer structure 116 to the fluid source 104. The transfer structure 116 can include one or more components. For example, the transfer structure 116 can be a single component having a surface configured to contact the fluid source 104. In some embodiments, the transfer structure 116 can include one or more members having a surface configured to contact the fluid source 104 upon activation of the electrochemical actuator 102. In some embodiments, the transfer structure 116 is a substantially planar or flat plate.

In some embodiments, the fluid delivery system 100 can be used to deliver a drug formulation which comprises a drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympnic, intraaural, topical, epidural, and/or peri-neural depending on, for example, the location of the fluid communicator 106 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Although the fluid delivery system 100 and other systems and methods described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may deliver drugs or other fluids into living organisms other than human beings, such as animals and plant life. Also, the systems and methods may deliver any fluids into any target, living or inanimate.

The systems and methods described herein are generally systems and methods of delivering fluids using a delivery device 100 that includes an electrochemical actuator 102, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Patent Pub. No. 2008/0257718, entitled “Electrochemical Actuator” by Chiang et al., and U.S. Patent Pub. No. 2009/0014320, entitled “Electrochemical Actuator” by Chiang et al., and U.S. Pat. No. 7,828,771, entitled “Systems and Methods for Delivering Drugs” by Chiang et al. (the '771 Patent), the disclosure of each of which is incorporated herein by reference. Such electrochemical actuators can include at least one component that responds to the application of a voltage or current by experiencing a change in volume or position. The change in volume or position can produce mechanical work that can then act on a fluid source (e.g., fluid source 104) or may be transferred to a fluid source, such that a fluid can be delivered out of the fluid source.

In some embodiments, the electrochemical actuator 102 can include a positive electrode and a negative electrode, at least one of which is an actuating electrode. These and other components of the electrochemical actuator can form an electrochemical cell, which can in some embodiments initially be charged. For example, the electrochemical cell may begin discharging when a circuit between the electrodes is closed, causing the actuating electrode to actuate. The actuating electrode can thereby perform work upon another structure, such as the fluid source, or a transfer structure associated with the fluid source, as described in more detail below. The work can then cause fluid to be pumped or otherwise dispensed from the fluid source into the target 108.

More specifically, the actuating electrode of the electrochemical actuator 102 can experience a change in volume or position when the closed circuit is formed, and this change in volume or position can perform work upon the fluid source or transferring structure. For example, the actuating electrode may expand, bend, buckle, fold, cup, elongate, contract, or otherwise experience a change in volume, size, shape, orientation, arrangement, or location, such that at least a portion of the actuating electrode experiences a change in volume or position. In some embodiments, the change in volume or position may be experienced by a portion of the actuating electrode, while the actuating electrode as a whole may experience a contrary change or no change whatsoever. It is noted that the delivery device 100 can include more than one electrochemical actuator 102. For example, in some embodiments, the delivery device 100 can include one or more electrochemical actuators 102 arranged in series, parallel, or some combination thereof. In some embodiments, a number of such electrochemical actuators 102 may be stacked together. As another example, concurrent or sequenced delivery of multiple agents can be achieved by including one or more electrochemical actuators 102 acting on two or more fluid sources.

The delivery system 100 can also include a housing (not shown in FIG. 1) that can be removably or releasably attached to the skin of the patient. The various components of the delivery system 100 can be fixedly or releasably coupled to the housing. To adhere the delivery device 100 to the skin of a patient, a releasable adhesive can at least partially coat an underside of the housing. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing can be formed from a material that is relatively lightweight and flexible, yet sturdy. The housing also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

In some embodiments, the housing can include a single component or multiple components. In some embodiments, the housing can include two portions: a base portion and a movable portion. The base portion can be suited for attaching to the skin. For example, the base portion can be relatively flexible. An adhesive can be deposited on an underside of the base portion, which can be relatively flat or shaped to conform to the shape of a particular body part or area. The movable portion can be sized and shaped for association with the base portion. In some embodiments, the two portions can be designed to lock together, such as via a locking mechanism. In some cases, the two portions can releasably lock together, such as via a releasable locking mechanism, so that the movable portion can be removably associated with the base portion. To assemble such a housing, the movable portion can be movable with reference to the base portion between an unassembled position and an assembled position. In the assembled position, the two portions can form a device having an outer shape suited for concealing the device under clothing. Various example embodiments of a housing are described in the '771 Patent incorporated by reference above.

The size, shape, and weight of the delivery device 100 can be selected so that the delivery device 100 can be comfortably worn on the skin after the device is applied via the adhesive. For example, the delivery device 100 can have a size, for example, in the range of about 1.0″×1.0″×0.1″ to about 5.0″×5.0″×1.0″, and in some embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml, and in some cases in the range of about 0.3 ml to about 100 ml, such as between about 0.5 ml and about 5 ml. The shape of the delivery device 100 can be selected so that the delivery device 100 can be relatively imperceptible under clothing. For example, the housing can be relatively smooth and free from sharp edges. However, other sizes, shapes, and/or weights are possible.

As mentioned above, an electrochemical actuator 102 can be used to cause the fluid delivery device 100 to deliver a drug-containing or non-drug containing fluid into a human patient or other target 108. Such a fluid delivery system 100 can be embodied in a relatively small, self-contained, and disposable device, such as a patch device that can be removably attached to the skin of a patient as described above. The delivery device 100 can be relatively small and self-contained because the electrochemical actuator 102 serves as both the battery and a pump. The small and self-contained nature of the delivery device 100 advantageously may permit concealing the device beneath clothing and may allow the patient to continue normal activity as the drug is delivered. Unlike conventional drug pumps, external tubing to communicate fluid from the fluid reservoir into the body can be eliminated. Such tubing can instead be contained within the delivery device, and a needle or other fluid communicator can extend from the device into the body. The electrochemical actuator 102 can initially be charged, and can begin discharging once the delivery device 100 is activated to pump or otherwise deliver the drug or other fluid into the target 108. Once the electrochemical actuator 102 has completely discharged or the fluid source 104 (e.g. reservoir) is empty, the delivery device 100 can be removed. The small and inexpensive nature of the electrochemical actuator 102 and other components of the device may, in some embodiments, permit disposing of the entire device after a single use. The delivery device 100 can permit drug delivery, such as subcutaneous or intravenous drug delivery, over a time period that can vary from several minutes to several days. Subsequently, the delivery device 100 can be removed from the body and discarded.

In use, the delivery device 100 can be placed in contact with the target 108 (e.g. placed on the surface of a patient's body), such that the fluid communicator 106 (e.g., a needle, cannula, etc.) is disposed adjacent to a desired injection site. The fluid communicator 106 can be actuated with the actuation of the electrochemical actuator 102 or separately as described in more detail below. For example, the delivery device 100 can include a separate mechanism to actuate the fluid communicator 106. Activation of the fluid communicator 106 can include, for example, insertion of the fluid communicator 106 into the patient's body. Example embodiments illustrating various configurations for actuation of the fluid communicator 106 are described in the '771 Patent. The electrochemical actuator 102 can then be actuated to apply a force on the fluid source 104, causing the fluid to be delivered through the fluid communicator 106 and into the target 108. For example, as the electrochemical actuator 102 is actuated, the actuator 102 will be displaced and will contact the amplification mechanism 118. As the amplification mechanism 118 is activated, the amplification mechanism 118 will apply a force to the transfer structure 116 and that force will in turn be transferred to the fluid source 104 to pump the fluid out of the fluid source 104, through the fluid communicator 106, and into the target 108.

Having described above various general principles, several exemplary embodiments of these concepts are now described. These embodiments are only examples, and many other configurations of a delivery system and/or the various components of a delivery system, are contemplated.

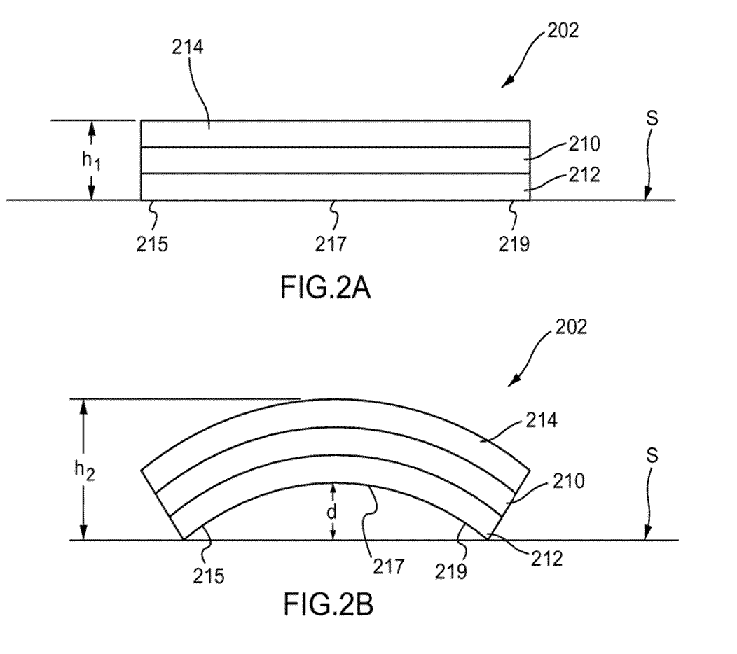

FIGS. 2A and 2B are schematic illustrations of an embodiment of an electrochemical actuator 202 that can be used in a delivery device as described herein. As shown, in this embodiment, the electrochemical actuator 202 can include a positive electrode 210, a negative electrode 212, and an electrolyte 214. These components can form an electrochemical cell that can initially be discharged and then charged before use, or can be initially charged, as shown in FIG. 2A. The positive electrode 210 can be configured to expand or displace in the presence of the electrolyte 214. When a circuit between the electrodes 210, 212 is closed, current can travel from the positive electrode 210 to the negative electrode 212. The positive electrode 210 can then experience a change in volume or shape, resulting in longitudinal displacement of at least a portion of the positive electrode 210, as shown in FIG. 2B. For example, the actuator 202 can have an overall height h₁ when it is charged (prior to actuation), as shown in FIG. 2A, and an overall height of h₂ when it is discharged or actuated, such that the actuator 202 has a displacement or stroke that is equal to h₂−h₁. Said another way, the actuator 202 can have a first end portion 215, a second end portion 219 and a medial portion 217 disposed between the first end portion 215 and the second end portion 219. The actuator prior to actuation (prior to discharge) can be supported on a surface S of the delivery device in which the actuator 202 is disposed, and when the actuator 202 is discharged at least the medial portion 217 can displace (e.g., bend or flex) a non-zero distance d from the surface S. The stroke of the actuator 202 can be substantially equal to that non-zero distance d. As the actuator 202 is displaced, the actuator 202 can exert a pumping force or pressure on a fluid reservoir (not shown) and/or an associated transfer structure (not shown) coupled thereto. The pumping force or pressure exerted by the actuator 202 can cause a volume of fluid (e.g., a therapeutic agent) to be pumped out of the fluid reservoir. Thus, the electrochemical actuator 202 can be considered a self-powered electrochemical pump.

In this embodiment, the electrochemical actuator 202 has a positive electrode 210 selected to have a lower chemical potential for the working ion when the electrochemical actuator 202 is charged, and is thereby able to spontaneously accept working ions from the negative electrode 212 as the actuator is discharged. In some embodiments, the working ion can include, but is not limited to, the proton or lithium ion. When the working ion is lithium, the positive electrode 210 can include one or more lithium metal oxides including, for example, LiCoO₂, LiFePO₄, LiNiO₂, LiMn₂O₄, LiMnO₂, LiMnPO₄, Li₄Ti₅O₁₂, and their modified compositions and solid solutions; oxide compound comprising one or more of titanium oxide, manganese oxide, vanadium oxide, tin oxide, antimony oxide, cobalt oxide, nickel oxide or iron oxide; metal sulfides comprising one or more of TiSi₂, MoSi₂, WSi₂, and their modified compositions and solid solutions; a metal, metal alloy, or intermetallic compound comprising one or more of aluminum, silver, gold, boron, bismuth, gallium, germanium, indium, lead, antimony, silicon, tin, or zinc; a lithium-metal alloy; or carbon comprising one or more of graphite, a carbon fiber structure, a glassy carbon structure, a highly oriented pyrolytic graphite, or a disordered carbon structure. The negative electrode 212 can include, for example, lithium metal, a lithium metal alloy, or any of the preceding compounds listed as positive electrode compounds, provided that such compounds when used as a negative electrode are paired with a positive electrode that is able to spontaneously accept lithium from the negative electrode when the actuator is charged. These are just some examples, as other configurations are also possible.

In some embodiments, the electrochemical actuator can include an anode, a cathode, and a species, such as a lithium ion. In some embodiments, a source of lithium ion is the electrolyte which is made up an organic solvent such as PC, propylene carbonate, GBL, gamma butyl lactone, dioxylane, and others, and an added electrolyte. Some example electrolytes include LiPF₆, LiBr, LiBF₄. At least one of the electrodes can be an actuating electrode that includes a first portion and a second portion. The portions can have at least one differing characteristic, such that in the presence of a voltage or current, the first portion responds to the species in a different manner than the second portion. For example, the portions can be formed from different materials, or the portions can differ in thickness, dimension, porosity, density, or surface structure, among others. The electrodes can be charged, and when the circuit is closed, current can travel. The species can, intercalate, de-intercalate, alloy with, oxide, reduce, or plate with the first portion to a different extent than the second portion. Due to the first portion responding differently to the species than the second portion, the actuating electrode can experience a change in one or more dimensions, volume, shape, orientation, or position.

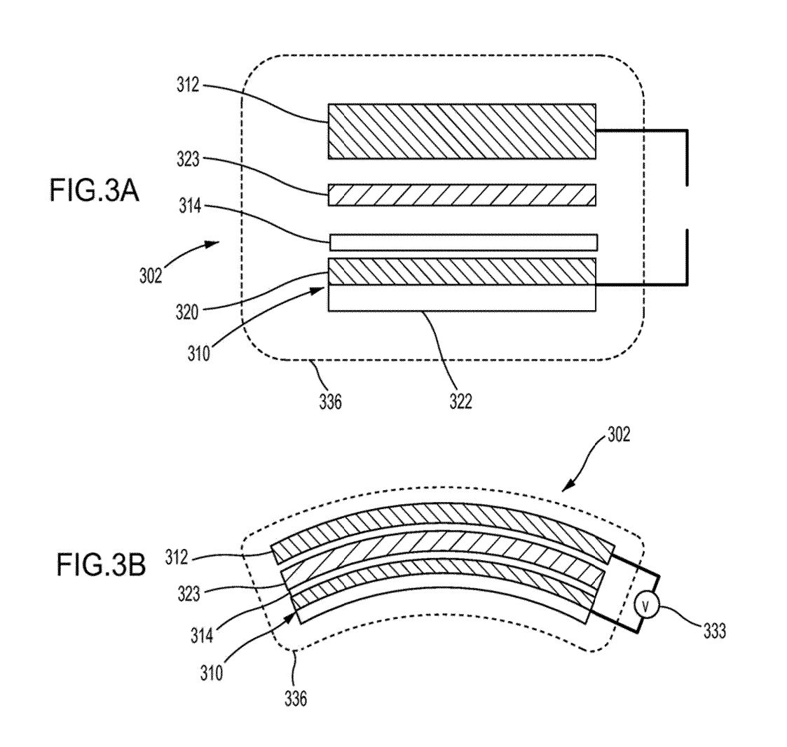

Another example of an electrochemical actuator is shown in the embodiment illustrated in FIGS. 3A and 3B. As shown in FIG. 3A, an electrochemical actuator 302 can include a negative electrode 312 in electrical communication with a positive electrode 310 collectively forming an electrochemical cell. Positive electrode 310 may include a first portion 320 and a second portion 322. In some embodiments, first portion 320 and second portion 322 are formed of different materials. Portions 320 and 322 may also have different electrical potentials. For example, first portion 320 may include a material that can intercalate, de-intercalate, alloy with, oxidize, reduce, or plate a species to a different extent than second portion 322. Second portion 322 may be formed of a material that does not substantially intercalate, de-intercalate, or alloy with, oxidize, reduce, or plate the species. In some embodiments, first portion 320 may be formed of a material including one or more of aluminum, antimony, bismuth, carbon, gallium, silicon, silver, tin, zinc, or other materials which can expand upon intercalation or alloying or compound formation with lithium. In one embodiment, first portion 320 is formed with aluminum, which can expand upon intercalation with lithium. Second portion 322 may be formed of copper, since copper does not substantially intercalate or alloy with lithium. In some instances, second portion 322 may act as a positive electrode current collector, and may extend outside the electrochemical cell, e.g., to form a tab or current lead. In other embodiments, second portion 322 may be joined to a tab or current lead that extends outside the cell. Negative electrode 312 may also include a current collector. Electrochemical actuator 302 may include a separator 323. The separator 323 may be, for example, a porous separator film, such as a glass fiber cloth, or a porous polymer separator. Other types of separators, such as those used in the construction of lithium ion batteries, may also be used. The electrochemical actuator 302 may also include an electrolyte 314, which may be in the form of a liquid, solid, or a gel. The electrolyte may contain an electrochemically active species, such as that used to form the negative electrode. Electrochemical actuator 302 may also include an enclosure 336, such as a polymer packaging, in which negative electrode 312, positive electrode 310 and separator 323 can be disposed.

As illustrated in FIG. 3B, the electrochemical cell may have a voltage 333, such that, when a closed circuit is formed between the negative electrode 312 and the positive electrode 310, an electric current may flow between the negative electrode 312 and the positive electrode 310 through the external circuit. If negative electrode 312 is a lithium metal electrode and the electrolyte contains lithium ions, lithium ion current can flow internally from the negative electrode 312 to the positive electrode 310. The intercalation of first portion 320 with lithium can result in a dimensional change, such as a volume expansion. In some instances, this volume expansion may reach at least 25%, at least 50%, at least 75%, at least 100%, at least 150%, at least 200%, at least 250%, or at least 300% compared to the initial volume. High volume expansion may occur, for example, when first portion 320 is saturated with lithium. As first portion 320 increases in volume due to intercalation of lithium, second portion 322 to which first portion 320 may be bonded, may not substantially expand due to minimal or no intercalation of lithium. First portion 320 thus provides a mechanical constraint. This differential strain between the two portions causes positive electrode 310 to undergo bending or flexure. As a result of the dimensional change and displacement of the positive electrode 310, electrochemical actuator 302 can be displaced from a first orientation to a second orientation. This displacement can occur whether the volumetric or dimensional change (e.g., net volume change) of the electrochemical cell, due to the loss of lithium metal from the negative electrode 312 and formation of lithium intercalated compound or lithium alloy at the positive electrode 310, is positive, zero, or negative. In some cases, the actuator displacement may occur with a volumetric or dimensional change (e.g., net volume change) of the electrochemical actuator 302, or portion thereof that is positive. In some cases, the actuator displacement may occur with a volumetric or dimensional change (e.g., net volume change) of the electrochemical actuator 302, or portion thereof that is zero. In some cases, the actuator displacement may occur with a volumetric or dimensional change (e.g., net volume change) of the electrochemical actuator 302, or portion thereof that is negative.

As used herein, “differential strain” between two portions can refer to the difference in response (e.g., actuation) of each individual portion upon application of a voltage or current to the two portions. That is, a system as described herein may include a component including a first portion and a second portion associated with (e.g., may contact, may be integrally connected to) the first portion, wherein, under essentially identical conditions, the first portion may undergo a volumetric or dimensional change and the second portion does not undergo a volumetric or dimensional change, producing strain between the first and second portions. The differential strain may cause the component, or a portion thereof, to be displaced from a first orientation to a second orientation. In some embodiments, the differential strain may be produced by differential intercalation, de-intercalation, alloying, oxidation, reduction, or plating of a species with one or more portions of the actuator system.

For example, the differential intercalation, de-intercalation, alloying, oxidation, reduction, or plating of first portion 320 relative to second portion 322 can be accomplished through several means. In one embodiment, first portion 320 may be formed of a different material than second portion 322, wherein one of the materials substantially intercalates, de-intercalates, alloys with, oxidizes, reduces, or plates a species, while the second portion interacts with the species to a lesser extent. In another embodiment, first portion 320 and second portion 322 may be formed of the same material. For example, first portion 320 and second portion 322 may be formed of the same material and may be substantially dense, or porous, such as a pressed or sintered powder or foam structure. In some cases, to produce a differential strain upon operation of the electrochemical cell, first portion 320 or second portion 322 may have sufficient thickness such that, during operation of the electrochemical cell, a gradient in composition may arise due to limited ion transport, producing a differential strain. In some embodiments, one portion or an area of one portion may be preferentially exposed to the species relative to the second portion or area of the second portion. In other instances, shielding or masking of one portion relative to the other portion can result in lesser or greater intercalation, de-intercalation, or alloying with the masked or shielded portion compared to the non-masked or shielded portion. This may be accomplished, for example, by a surface treatment or a deposited barrier layer, lamination with a barrier layer material, or chemically or thermally treating the surface of the portion to be masked/shielded to either facilitate or inhibit intercalation, de-intercalation, alloying, oxidation, reduction, or plating with the portion. Barrier layers can be formed of any suitable material, which may include polymers, metals, or ceramics. In some cases, the barrier layer can also serve another function in the electrochemical cell, such as being a current collector. The barrier layer may be uniformly deposited onto the surface in some embodiments. In other cases, the barrier layer may form a gradient in composition and/or dimension such that only certain portions of the surface preferentially facilitate or inhibit intercalation, de-intercalation, alloying, oxidation, reduction, or plating of the surface. Linear, step, exponential, and other gradients are possible. In some embodiments a variation in the porosity across first portion 320 or second portion 322, including the preparation of a dense surface layer, may be used to assist in the creation of an ion concentration gradient and differential strain. Other methods of interaction of a species with a first portion to a different extent so as to induce a differential strain between the first and second portions can also be used. In some embodiments, the flexure or bending of an electrode is used to exert a force or to carry out a displacement that accomplishes useful function.

In some embodiments, the electrical circuit can include electrical contacts (not shown) that can open or close the electrical circuit. For example, when the electrical contacts are in communication with each other, the electrical circuit will be closed (as shown in FIG. 3B) and when they are not in contact with each other, the electrical circuit can be opened or broken, as shown in FIG. 3A.

The discharge of the electrochemical actuator can be relatively proportional to the current traveling through the electrical circuit (i.e., the electrical resistance of the resistor). Because the electrical resistance of the resistor can be relatively constant, the electrochemical actuator can discharge at a relatively constant rate. Thus, the discharge of the electrochemical actuator, and thus the displacement of the electrochemical actuator can be relatively linear with the passage of time.

In some embodiments, an electrical circuit can be used that includes a variable resistor. By varying the resistance, the discharge rate of the electrochemical actuator and the corresponding displacement of the electrochemical actuator can be varied, which in turn can vary the fluid flow rate from the fluid source. An example of such an embodiment is described in the '771 Patent. In some embodiments, an electrical circuit can be used that uses a switch to open or close the electrical circuit. When the switch is closed, the electrochemical actuator can discharge and when the switch is opened, the electrochemical actuator can be prevented from discharging. An example of such an embodiment is described in the '771 Patent incorporated by reference above.

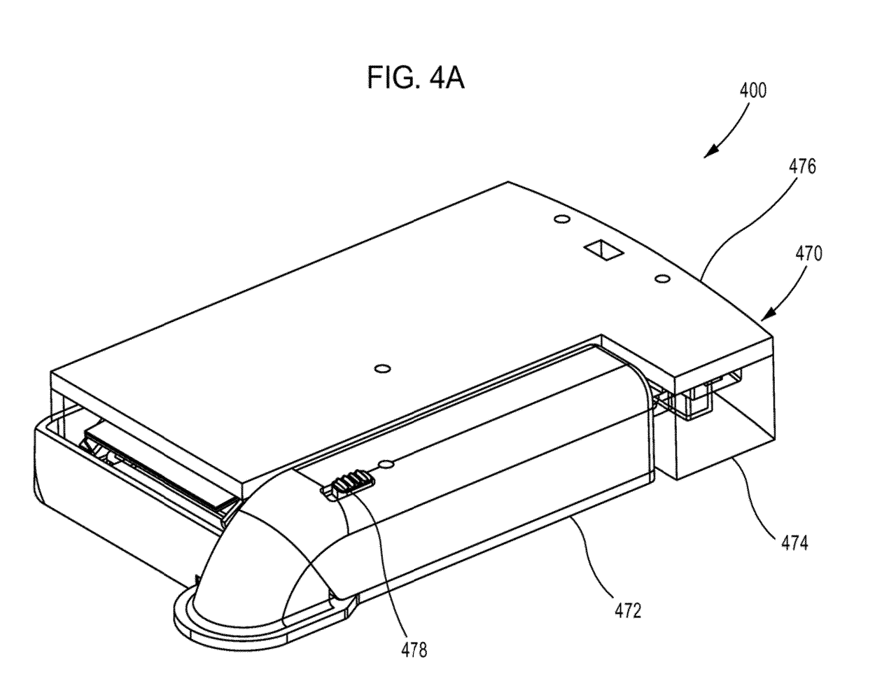

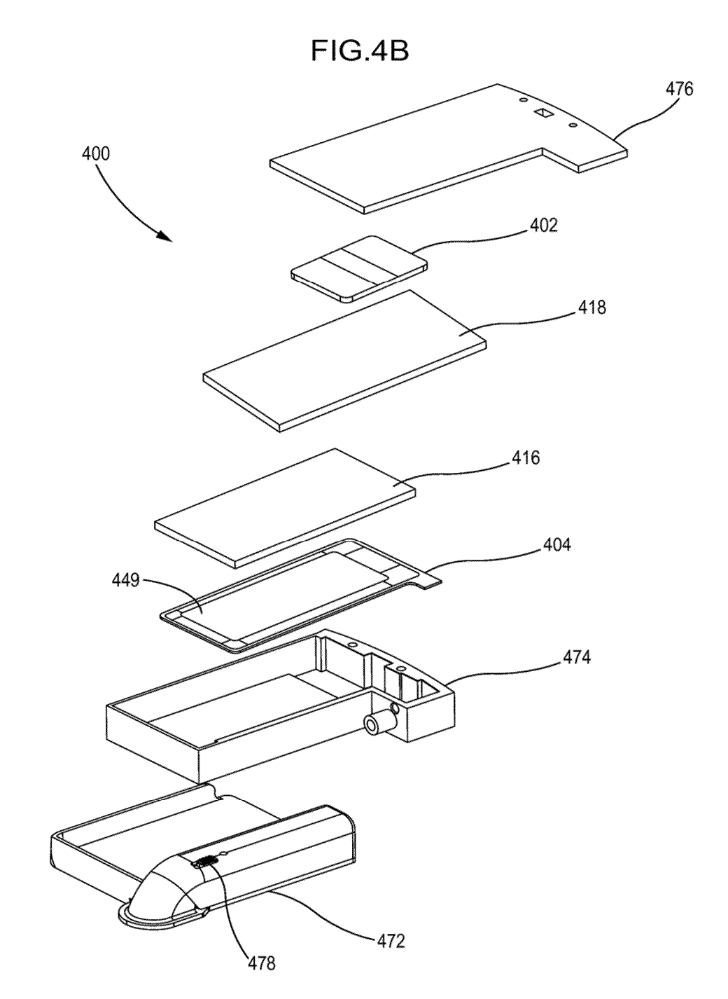

FIGS. 4A and 4B illustrate an embodiment of a delivery device that can include an electrochemical actuator as described herein. A delivery device 400 includes a housing 470, a fluid source 404, an electrochemical actuator 402, an amplification mechanism 418 (shown schematically in FIG. 4B), optionally, a transfer structure 416 can be disposed between the fluid source 404 and the actuator 402, and associated electronics (not shown) that can be coupled to the electrochemical actuator 402. In this embodiment, the housing 470 includes a first portion 472, a second portion 474, and a top portion 476 that can be coupled together to form an interior region within the housing 470. The fluid source 404, the electrochemical actuator 402, the amplification mechanism 418 and the transfer structure 416 can each be disposed within the interior region defined by the housing 470.

The fluid source 404 can be provided to a user already disposed within the interior region of the housing 470 or can be provided as a separate component that the user can insert into the housing 470. For example, the fluid source 404 can be inserted through an opening (not shown) in the housing 470. The fluid source 404 can be, for example, a fluid reservoir, bag or container, etc. that defines an interior volume that can contain a fluid to be injected into a patient. The fluid source 404 (also referred to herein as “fluid reservoir”) can include a web portion (not shown) configured to be punctured by an insertion mechanism (not shown) to create a fluid channel between the fluid source 404 and a fluid communicator (not shown) configured to penetrate the patient's skin. In some embodiments, the fluid reservoir 404 can be sized for example, with a length L of about 2 cm, a width W of about 2 cm, and a height H of about 0.25 cm, to contain, for example, a total volume of 1 ml of fluid.

The delivery device 400 also includes an activation mechanism 478 in the form of button that can be used to activate the insertion mechanism and/or the actuator 402. The first portion 472, the second portion 474 and the top portion 476 of the housing 470 can be coupled together in a similar manner as with various embodiments of a delivery system described in the '771 Patent incorporated by reference above. The first portion 472, the second portion 474 and the top portion 476 can be coupled, for example, with an adhesive, a snap fit coupling or other known coupling method. The first portion 472 can be adhered to a patient's body with an adhesive layer disposed on a bottom surface of the first portion 472.

To use the delivery device 400, the delivery device 400 is placed at a desired injection site on a patient's body and adhesively attached thereto. When the fluid source 404 is disposed within the housing 470 (e.g., inserted into the housing by the patient or predisposed), the activation mechanism 478 (e.g., button, switch, lever, pull-tab, etc.) can be moved from an off position to an on position, which will cause the fluid communicator to penetrate the patient's skin at the treatment site. Alternatively, in some embodiments, the insertion mechanism (not shown) can be activated by the fluid source 404 being inserted into the housing.

The electrochemical actuator 402 can be activated after the insertion mechanism has been activated and the fluid communicator is inserted into the patient's body. Alternatively, in some embodiments, the electrochemical actuator 402 can be activated simultaneously with activation of the insertion mechanism. For example, when the insertion mechanism is activated it can be configured to activate a trigger mechanism (not shown) that communicates with the electrochemical actuator 402. For example, such a trigger mechanism can complete the electric circuit (as described above) and cause the electrochemical actuator 402 to start discharging. As the electrochemical actuator 402 discharges, the actuator 402 and the amplification mechanism 418 will displace and exert a force on the transfer structure 416, which in turn will exert a force on the top surface 449 of the fluid source 404, thereby compressing the fluid source 404 between the transfer structure 416 and the second portion 474 of the housing 470 and causing a volume of fluid within the fluid source 404 to be expelled into the patient.

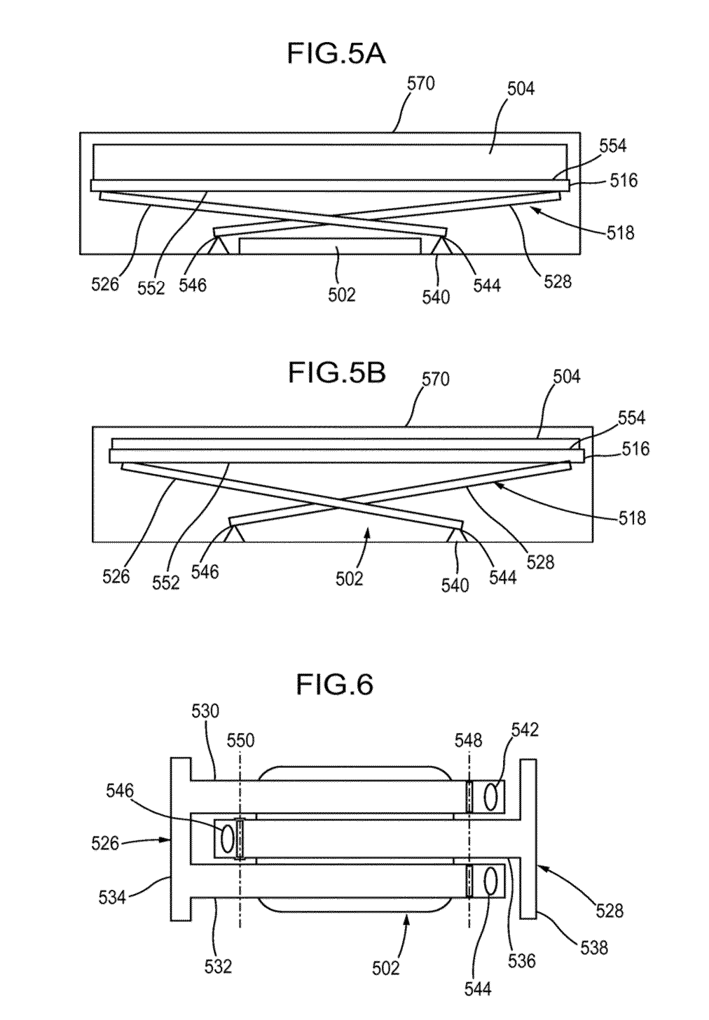

FIGS. 5A, 5B and 6 are schematic illustrations of a portion of an embodiment of a delivery system illustrating an amplification mechanism that can be used to amplify or enhance the displacement, force and/or change the duration of the drug delivery. FIG. 5A is a side view showing a housing 570, a fluid reservoir 504, a transfer structure 516, an amplification mechanism 518, and an electrochemical actuator 502 in a charged state prior to actuation, each disposed within the housing 570; and FIG. 5B is a side view showing the housing 570, the fluid reservoir 504, the transfer structure 516, the amplification mechanism 518, and the electrochemical actuator 502 with a portion of the electrochemical actuator 502 in a discharged or displaced configuration. The housing 570 can be configured the same as or similar to the housing described above for delivery device 100. For example, the housing can be removably or releasably attached to the skin of the patient with for example, an adhesive. The fluid reservoir 504 can contain a volume of fluid (e.g., a therapeutic agent) therein prior to the actuator 502 being discharged, as shown in FIG. 5A. FIG. 6 is a top view showing only the amplification mechanism 518 and the electrochemical actuator 502. Although not shown in the side views of FIGS. 5A and 5B, the transfer structure 516 in this embodiment is in the form of a substantially planar plate.

In this embodiment, the amplification mechanism 518 includes two independently movable levers: a first lever 526 and a second lever 528. The first lever 526 includes a first arm 530, a second arm 532, and a push bar 534. The second lever 528 includes a single arm 536 and a push bar 538. The first lever 526 is attached to a base 540 at an anchor location 542 and an anchor location 544 and the second lever 528 is attached to the base 540 at an anchor location 546, as shown in FIG. 6. The first lever 526 and the second lever 528 can be attached to the base 540, for example, with pins or other known mechanical attachment, with adhesive, or can be molded thereto. The push bars 534 and 538 can each contact a bottom surface 552 of the transfer structure 516 shown in FIGS. 5A and 5B. The fluid reservoir 504 shown in FIGS. 5A and 5B can be disposed adjacent a top surface 554 of the transfer structure 516.

As the electrochemical actuator 502 is actuated such that a portion of the actuator 502 is displaced as described above (and as shown in FIG. 5B, the actuator 502 will contact and move the levers 526 and 528 upward, which will each pivot upward at a pivot axis 548 and a pivot axis 550, respectively. In this embodiment, the amplification mechanism 518 is a double cantilever arrangement where the amplification can be determined by the relative distance to the pivot points of the electrochemical actuator 502 from the anchor locations of the respective arms 530, 532, 536. As the levers 526 and 528 are pivoted upward, the push bar 534 and the push bar 538 will in turn move the transfer structure 516 upward. The transfer structure 516 will then exert a force on the fluid reservoir 504 as described previously to pump the volume of fluid out of the fluid reservoir 504 and into a target body.

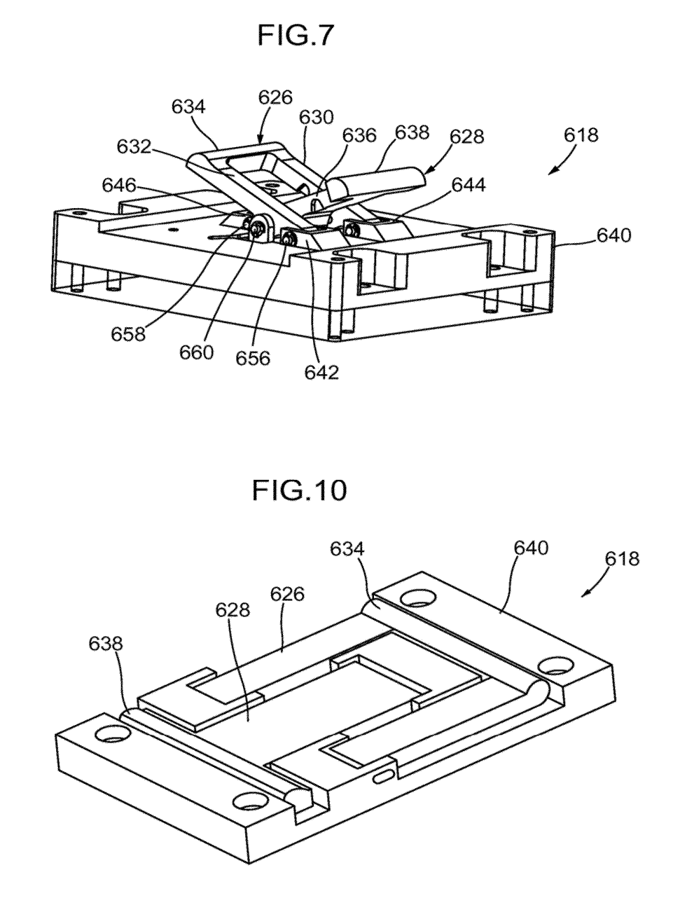

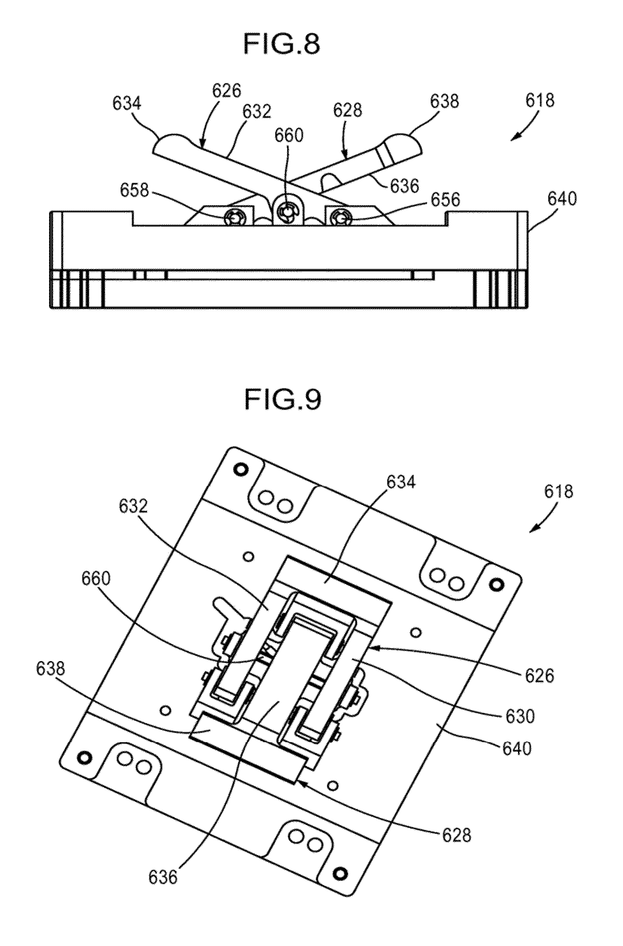

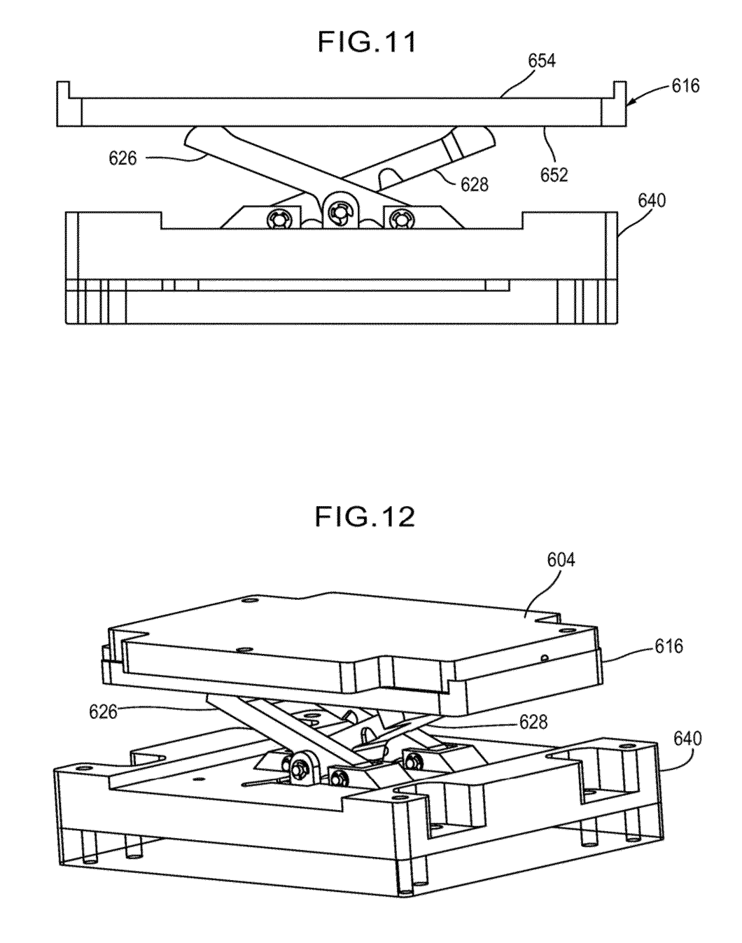

FIGS. 7-12 illustrate another embodiment of an amplification mechanism that can be used in a delivery system as described herein. An amplification mechanism 618 includes a first lever 626 and a second lever 628. The first lever 626 includes a first arm 630, a second arm 632, and a push bar 634. The second lever 628 includes a single arm 636 and a push bar 638. In this embodiment, the first lever 626 is coupled to a base 640 at a mounting location 642 and a mounting location 644 with a pin 656. The second lever 628 is coupled to the base 640 at a mounting location 646 with a pin 658. The first lever 626 and the second lever 628 are also coupled to each other with a pin 660. One or both of the levers 626 and 628 can also be slidably coupled to the base 640. In other words, one or both of levers 626 and 628 can pivot or rotate about its mounting location and also slide relative to the base 640. The push bars 634 and 638 can each contact a bottom surface 652 of a transfer structure 616 and a fluid source 604 (see FIG. 12) can be disposed adjacent a top surface 654 (see FIG. 11) of the transfer structure 616.

An electrochemical actuator (not shown) can be disposed beneath the levers 626 and 628. Prior to activation of the actuator, the levers 626 and 628 can be in a collapsed or folded configuration, as shown in FIG. 10. As described previously, when the electrochemical actuator is activated, a portion of the actuator will be displaced as described above, such that the actuator will contact the levers 626 and 628, which will each pivot about its respective mounting location. As the levers 626 and 628 are pivoted upward, the push bar 634 and the push bar 638 will in turn move the transfer structure 616 upward as shown in FIGS. 11 and 12. The transfer structure 616 will then exert a force on the fluid source 604 as described previously to pump the fluid out of the fluid source and into a target body.

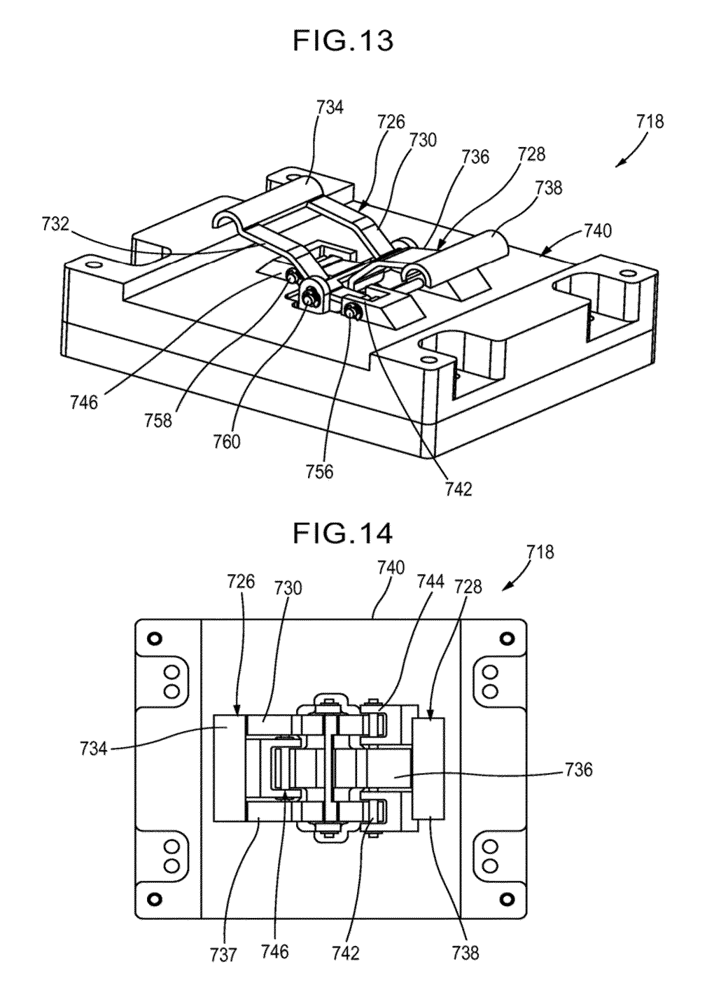

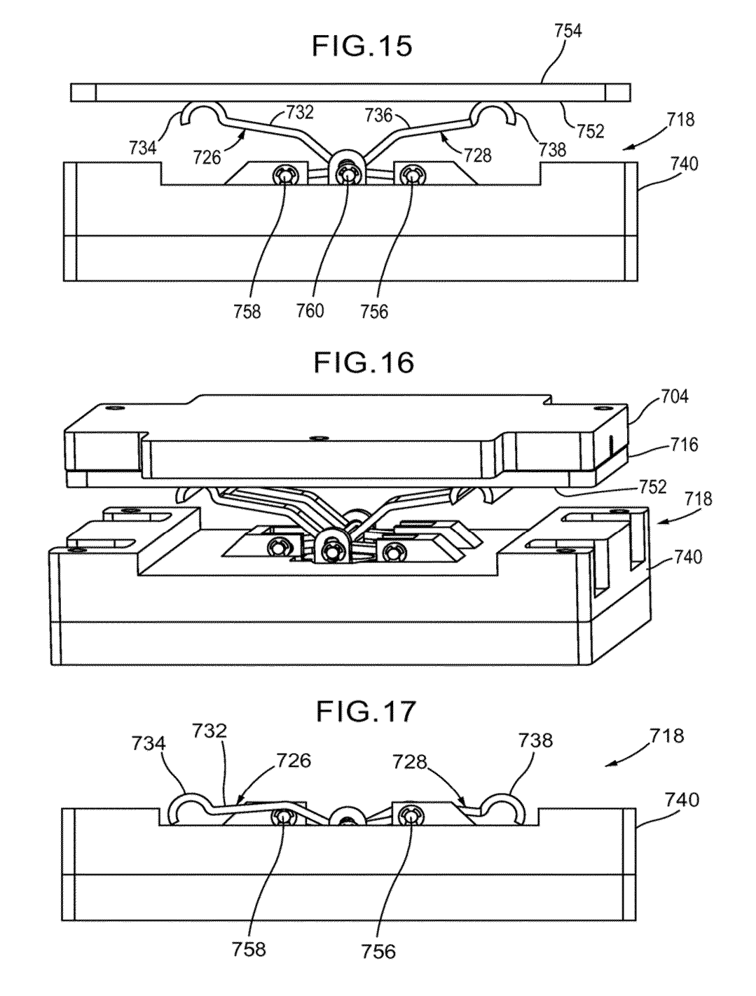

FIGS. 13-17 illustrate another embodiment of an amplification mechanism that can be used in a delivery system as described herein. This embodiment illustrates an amplification mechanism having levers with reduced thickness and also with a shape that illustrates a design that can be routed around pins and other features within the delivery device. An amplification mechanism 718 includes a first lever 726 and a second lever 728. The first lever 726 includes a first arm 730, a second arm 732, and a push bar 734. The second lever 728 includes a single arm 736 and a push bar 738. In this embodiment, the first lever 726 is coupled to a base 740 at a mounting location 742 and a mounting location 744 with a pin 756. The second lever 728 is coupled to the base 740 at a mounting location 746 with a pin 756. The first lever 726 and the second lever 728 are also coupled to each other with a pin 760. One or both of the levers 726 and 728 can also be slidably coupled to the base 740. In other words, one or both of levers 626 and 628 can pivot or rotate about its mounting location and also slide relative to the base 740. The push bars 734 and 738 can each contact a bottom surface 752 of a transfer structure 716 and a fluid source 704 can be disposed adjacent a top surface 754 (see FIG. 15) of the transfer structure 716 as shown in FIG. 16.

An electrochemical actuator (not shown) can be disposed beneath the levers 726 and 728. Prior to activation of the actuator, the levers 726 and 728 can be in a collapsed or folded configuration as shown in FIG. 17. As described previously, when the electrochemical actuator is activated, a portion of the actuator will be displaced as described above, such that the actuator 702 will contact the levers 726 and 728, which will each pivot about its respective mounting location. As the levers 726 and 728 are pivoted upward, the push bar 734 and the push bar 738 will in turn move the transfer structure 716 upward as shown in FIGS. 15 and 16. The transfer structure 716 will then exert a force on the fluid source 704 as described previously to pump the fluid out of the fluid source and into a target body.

As discussed above, an amplification mechanism (e.g., 518, 618, 718) can amplify the motion of the electrochemical actuator of a drug delivery system. The pin 660 (760) attachment of the levers 626, 628 (726, 728) is not required, but can be used to ensure that the motion from the electrochemical actuator that is applied to one lever can be transferred to the other lever. In some embodiments, the actuator can engage both levers at the same time ensuring that equal motion is captured by both levers. The mounting locations 642, 644, 646 (742, 744, 746) for the levers 626 and 628 (726 and 728) ensure that the push bars 634, 638 (734, 738) can move in unison in a plane parallel to the base 640 (740). The u-shape of the lever 626 (726) and the t-shape of the lever 628 (728) allows for the levers 626 and 628 (726 and 728) to interlock in the collapsed or folded configuration (see, e.g., FIG. 10) and minimize space requirements in the drug delivery device. The mechanical motion amplification can be determined, at least in part, on a ratio of a length of the levers 626, 628 (726, 728) to a distance of motion input from the respective mounting locations 642, 644, 646 (742, 744, 746). In addition, the force available at the push bars 634, 638 (734, 738) can be reduced by the same ratio.

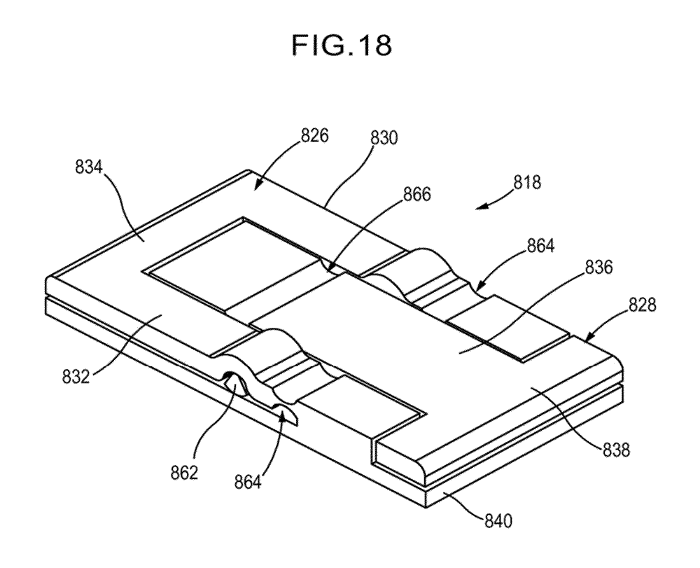

FIG. 18 is a perspective view of another embodiment of an amplification mechanism that can be formed as a single injection molded component. An amplification mechanism 818 (shown in collapsed or folded configuration) includes a first lever 826, a second lever 828 and a based 840. The first lever 826 includes a first arm 830, a second arm 832, and a push bar 834. The second lever 828 includes a single arm 836 and a push bar 838. In this embodiment, as mentioned above, the amplification mechanism 818 is formed as a single injection molded component, therefore the levers 826 and 828 are not coupled to the base 840 with a pin as in the previous embodiments. The motion of the levers 826 and 828 is achieved in part by an integrally formed pin-type appendix 862 that contacts the arms 832 and 830 of the lever 826 and a similar appendix (not shown) that contacts the arm 836 of the lever 828. A flexing motion also occurs at a necked-down or narrow portion 864 of the arms 830 and 832 and a necked-down or narrow portion 866 of the arm 836. It should be understood, however, that a pin through lever 836 (with hole properly molded or otherwise formed) can also be used.

The push bars 834 and 838 can each contact a bottom surface of a transfer structure (not shown) and a fluid source (not shown) can be disposed adjacent a top surface of the transfer structure in a similar manner as described above for previous embodiments. An electrochemical actuator (not shown) can be disposed beneath the levers 826 and 828. Prior to activation of the actuator, the levers 826 and 828 can be in a collapsed or folded configuration, as shown in FIG. 18, and when the electrochemical actuator is activated, the levers 826 and 828 can be moved upward as described previously. Thus, the amplification mechanism 818 can amplify the force and/or displacement of the actuator as described previously to pump fluid out of a fluid source and into a target body.

In alternative embodiments, an injection molded version of an amplification mechanism can be formed as two or more components. For example, each of the levers can be molded as a separate component and a through-hole can be provided to accommodate a common pin to couple the levers together.

FIGS. 19 and 20 are schematic illustrations of an electrochemical actuator 902 and an amplification mechanism 918, shown charged and discharged, respectively. The electrochemical actuator 902 can be configured the same as or similar to any of the embodiments of an electrochemical actuator described herein and the amplification mechanism 918 can be configured the same as or similar to any of the embodiments of an amplification mechanism described herein. The electrochemical actuator 902 and amplification mechanism 918 can be used in a delivery device as described herein. As shown in FIG. 19, when in a charged state (e.g., prior to actuation), the electrochemical actuator 902 can have a height H₁ and the amplification mechanism can have a height h₁. As shown in FIG. 20, when the electrochemical actuator 902 discharged or actuated, the electrochemical actuator 902 can be displaced to a height of H₂ and the amplification mechanism 918 can also be displaced to a height h₂. The overall displacement is equal to the displacement of the actuator (H₂−H₁) plus the displacement of the amplification mechanism (h₂−h₁).

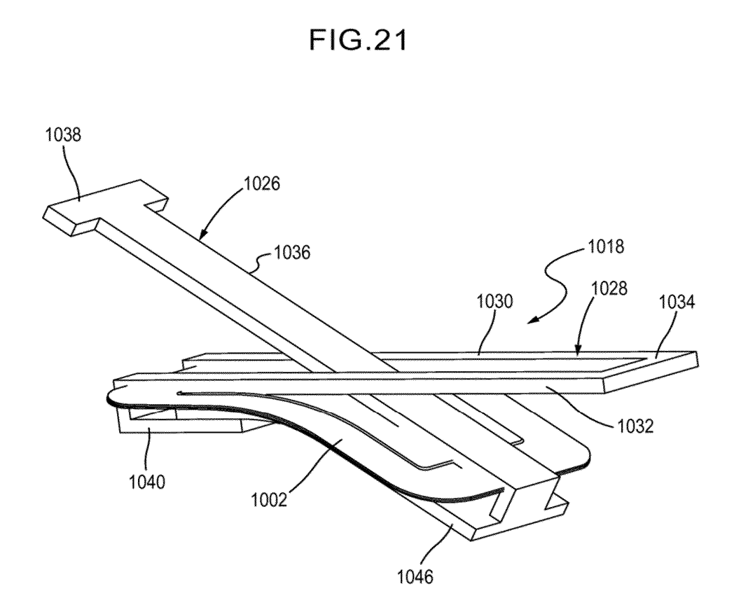

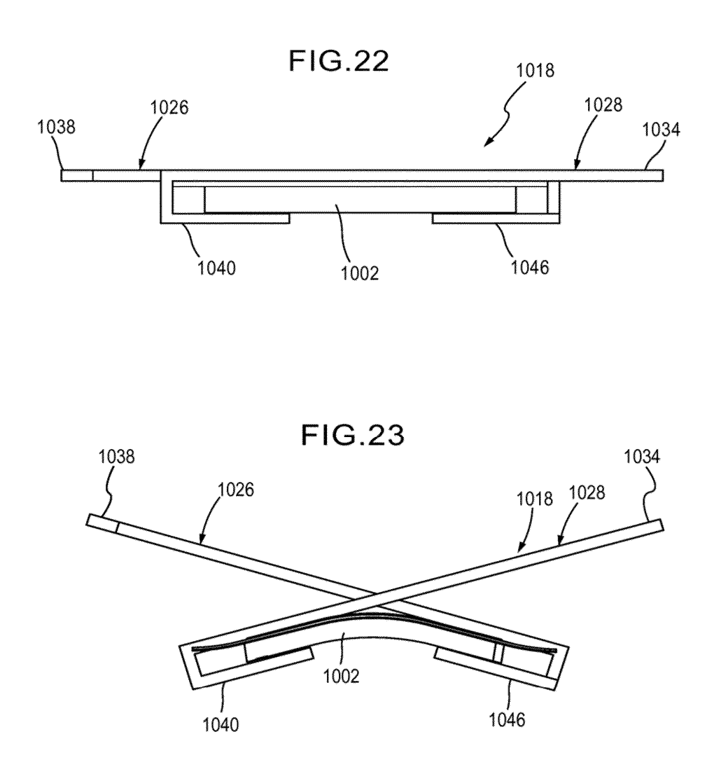

FIGS. 21-23 illustrate an embodiment of an electrochemical actuator and an integrally formed amplification mechanism that can be used, for example, in a drug delivery device. An amplification mechanism 1018 includes a first lever 1026 and a second lever 1028. The first lever 1026 includes a single arm 1036, a push bar 1038 and a base portion 1046. The second lever 1028 includes a first arm 1030, a second arm 1032, a push bar 1034 and a base portion 1040. In this embodiment, as mentioned above, the amplification mechanism 1018 is integrally formed in a single piece, e.g. by injection molding. The electrochemical actuator 1002 can be disposed and retained in operative engagement with the amplification mechanism 1018. The levers 1026 and 1028 are formed with a flexible material such that the levers 1026 and 1028 can flex or pivot upward as the electrochemical actuator 1002 displaces (e.g., bends) as shown, for example, in FIGS. 21 and 24. In alternative embodiments, the levers 1026 and 1028 are formed with rigid material such that levers 1026 and 1028 can pivot upward as the electrochemical actuator 1002 displaces. FIG. 22 illustrates the amplification mechanism 1018 in a collapsed or pre-activated configuration, and FIGS. 21 and 23 illustrate the amplification mechanism 1018 and actuator 1002 in an extended configuration.

Prior to activation of the actuator 1002, the levers 1026 and 1028 of the amplification mechanism 1018 are in a collapsed or folded configuration, as shown in FIG. 22. When the electrochemical actuator 1002 is activated (as described for other embodiments), the amplification mechanism 1018 will flex upward and the push bars 1034 and 1038 of the amplification mechanism 1018 can each contact a bottom surface of a transfer structure (not shown), which can in turn contact and exert a force on a fluid source (not shown) in a similar manner as described above for previous embodiments to push a volume of fluid out of the fluid source and into a patient.

A delivery device (e.g., 100, 900) as described herein may be used to deliver a variety of drugs according to one or more release profiles. For example, the drug may be delivered according to a relatively uniform flow rate, a varied flow rate, a preprogrammed flow rate, a modulated flow rate, in response to conditions sensed by the device, in response to a request or other input from a user or other external source, or combinations thereof. Thus, embodiments of the delivery device may be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

For example, the delivery devices may be useful in a wide variety of therapies. Representative examples include, but are not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine, and bupivicaine; dopamine antagonists such as apomorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline. Essentially any peptide, protein, biologic, or oligonucleotide, among others, that is normally delivered by subcutaneous, intramuscular, or intravenous injection or other parenteral routes, may be delivered using embodiments of the devices described herein. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, the delivery device may be used to administer ketamine for the treatment of refractory depression or other mood disorders. In some embodiments, ketamine may include either the racemate, single enantiomer (R/S), or the metabolite (wherein S-norketamine may be active). In some embodiments, the delivery devices described herein may be used for administration of Interferon A for the treatment of hepatitis C. In one embodiment, a several hour infusion patch is worn during the day or overnight three times per week, or a continuous delivery system is worn 24 hours per day. Such a delivery device may advantageously replace bolus injection with a slow infusion, reducing side effects and allowing the patient to tolerate higher doses. In other Interferon A therapies, the delivery device may also be used in the treatment of malignant melanoma, renal cell carcinoma, hairy cell leukemia, chronic hepatitis B, condylomata acuminata, follicular (non-Hodgkin's lymphoma, and AIDS-related Kaposi's sarcoma.

In some embodiments, a delivery device as described herein may be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. The delivery devices described herein may provide continuous delivery and may dramatically reduce side effects associated with both apomorphine and dopamine fluctuation. In some embodiments, a delivery device as described herein can provide continuous delivery of apomorphine or other dopamine agonist, with, optionally, an adjustable baseline and/or a bolus button for treating an “off” state in the patient. Advantageously, this method of treatment may provide improved dopaminergic levels in the body, such as fewer dyskinetic events, fewer “off” states, less total time in “off” states, less cycling between “on” and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

### CONCLUSION

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. The specific configurations of the various components can also be varied. For example, the size and specific shape of the various components can be different than the embodiments shown, while still providing the functions as described herein. In addition, although the amplification mechanism was described herein with reference to use with particular embodiments of a drug delivery device, an amplification mechanism can also be included in other embodiments of a drug delivery device to enhance or amplify the force and/or displacement of an actuator.

What is claimed is:

1. An apparatus, comprising:
a reservoir configured to contain a fluid;
a fluid communicator configured to be placed in fluid communication with the reservoir;
an actuator configured to be actuated such that a force is exerted on the reservoir for a time period upon actuation such that fluid within the reservoir is communicated through the fluid communicator; and
an amplification mechanism operatively coupled to the actuator, the amplification mechanism configured to at least one of increase the force exerted by the actuator on the reservoir or increase an overall stroke of the apparatus.

2. The apparatus of claim 1, further comprising:
a transfer structure disposed between the amplification mechanism and the reservoir, the transfer structure configured to contact the reservoir upon actuation of the actuator.

3. The apparatus of claim 1, wherein the amplification mechanism includes a first lever and a second lever, the first lever and the second lever each configured to rotate about a pivot location upon actuation of the actuator.

4. The apparatus of claim 1, wherein the amplification mechanism includes a base and a lever coupled to the base, the lever including a first end pivotally coupled to the base and a push bar disposed at a second end of the lever, the lever configured to rotate when the actuator is moved from its first configuration to its second configuration such that a top surface of the push bar is moved away from the base and maintains an orientation parallel to a top surface of the base.

5. The apparatus of claim 1, wherein the actuator is an electrochemical actuator.

6. The apparatus of claim 1 where the amplification mechanism is integrally formed with the actuator.

7. An apparatus, comprising:
a reservoir configured to contain a fluid;
an actuator having a first end portion, a second end portion and a medial portion between the first end portion and the second portion, the actuator having a first configuration in which the medial portion of the actuator contacts a support surface within the apparatus and a second configuration in which the medial portion of the actuator is disposed at a non-zero distance from the support surface, the actuator configured to exert a force on the reservoir when the actuator moves from its first configuration to its second configuration such that fluid within the reservoir is communicated out of the fluid reservoir; and
an amplification mechanism operatively coupled to the actuator and disposed between the actuator and the fluid reservoir, the amplification mechanism having a first configuration in which the amplification mechanism has a first height and a second configuration in which the amplification mechanism has a second height greater than its first height.

8. The apparatus of claim 7, wherein the actuator has a stroke equal to the non-zero distance from the support surface, the amplification mechanism having a stroke equal to a difference between the second height of the amplification mechanism and the first height of the amplification mechanism.

9. The apparatus of claim 7, wherein the apparatus has a stroke defined by a sum of a stroke associated with the actuator and a stroke associated with the amplification mechanism.

10. The apparatus of claim 7, further comprising:
a transfer structure disposed between the amplification mechanism and the reservoir, the transfer structure configured to contact the reservoir when the actuator is moved from its first configuration to its second configuration.

11. The apparatus of claim 7, wherein the amplification mechanism includes a first lever and a second lever, the first lever and the second lever each configured to rotate about a pivot location when the actuator is moved from its first configuration to its second configuration.

12. The apparatus of claim 7, wherein the actuator is an electrochemical actuator.

13. The apparatus of claim 7 where the amplification mechanism is integrally formed with the actuator.

14. An apparatus, comprising:
a reservoir configured to contain a fluid;
an actuator having a first configuration and a second configuration, the actuator configured to exert a force on the reservoir when the actuator moves from its first configuration to its second configuration such that fluid within the reservoir is communicated out of the fluid reservoir; and
an amplification mechanism operatively coupled to the actuator and disposed between the actuator and the fluid reservoir, the amplification mechanism having a first configuration in which the amplification mechanism has a first height and a second configuration in which the amplification mechanism has a second height greater than its first height,
the actuator when moved from its first configuration to its second configuration defining a first stroke, the amplification mechanism when moved from its first configuration to its second configuration defining a second stroke, a stroke of the apparatus being collectively defined by a sum of the first stroke and the second stroke.

15. The apparatus of claim 14, wherein the actuator when in its first configuration contacts a support surface within the apparatus, the actuator when in its second configuration being disposed at a non-zero distance from the support surface, the stroke of the actuator being equal to the non-zero distance.

16. The apparatus of claim 14, wherein the amplification mechanism has a first height when in its first configuration and a second height when in its second configuration, the stroke of the amplification mechanism being equal to a difference between the second height of the amplification mechanism and the first height of the amplification mechanism.

17. The apparatus of claim 14, further comprising:
a transfer structure disposed between the amplification mechanism and the reservoir, the transfer structure configured to contact the reservoir when the actuator is moved from its first configuration to its second configuration.

18. The apparatus of claim 14, wherein the amplification mechanism includes a first lever and a second lever, the first lever and the second lever each configured to rotate about a pivot location when the actuator is moved from its first configuration to its second configuration.

19. The apparatus of claim 14, wherein the actuator is an electrochemical actuator.

20. The apparatus of claim 14, where the amplification mechanism is integrally formed with the actuator.

21. An apparatus for delivering a therapeutic to a patient comprising:
a reservoir configured to contain a fluid; and
a fluid delivery mechanism, the fluid delivery mechanism including an electrochemical actuator including an electrode configured to displace as the electrochemical actuator undergoes a change in electrical potential, the displacement of the electrode being operative to exert a first force on an amplification mechanism, the amplification mechanism configured to exert a second force, different than the first force, on the reservoir in response to the first force exerted by the displacement of the electrode such that fluid is communicated out of the fluid reservoir.

22. The apparatus of claim 21, wherein the first force is greater than the second force.

23. The apparatus of claim 21, wherein the second force is greater than the first force.

24. The apparatus of claim 21, further comprising:
a transfer structure disposed between the amplification mechanism and the fluid reservoir, the second force being exerted on the fluid reservoir via the transfer structure.

25. The apparatus of claim 21, wherein the electrochemical actuator has a first end portion, a second end portion and a medial portion between the first end portion and the second end portion, the electrochemical actuator having a first configuration prior to the displacement of the electrode in which the medial portion of the electrochemical actuator contacts a support surface within the apparatus, and a second configuration after the displacement of the electrode in which the medial portion of the electrochemical actuator is at a non-zero distance from the support surface.
